(19) United States
(12) Patent Application Publication
(10) Pub. No.: US 2024/0009405 A1
(43) Pub. Date: Jan. 11, 2024

(54) NEEDLE AND SYRINGE USING SAME

(71) Applicant: INDUSTRY-ACADEMIC COOPERATION FOUNDATION, YONSEI UNIVERSITY, Seoul (KR)

(72) Inventors: Seong Taek Kim, Seoul (KR); Joon Sang Lee, Seoul (KR)

(21) Appl. No.: 18/254,494

(22) PCT Filed: Nov. 18, 2021
(86) PCT No.: PCT/KR2021/016962

§ 371 Date: May 25, 2023

(30) Foreign Application Priority Data
Nov. 25, 2020 (KR) 10-2020-0159923

(51) Int. Cl. A61M 5/32

(52) U.S. Cl. CPC A61M 5/3291

(57) ABSTRACT

A needle and a syringe using the same according to the present disclosure include a needle provided in a device for injecting a medicinal liquid through a skin layer, and including outlets formed along the longitudinal direction of the needle between one end of the needle connected to an accommodating part of the device containing the medicinal liquid and the other end of the needle for penetrating the skin layer, so that the medicinal liquid is evenly injected into a skin tissue in the lower portion of the skin layer, wherein the outlets are formed in a first section between the other end and a location corresponding to a tendon membrane located inside the skin tissue in a state of penetrating the skin layer, and a second section between the location corresponding to the tendon membrane and the one end, respectively, so that the medicinal liquid is evenly injected around the tendon membrane, and a syringe using such needle is also provided.

### TECHNICAL FIELD

The present disclosure relates to a needle and a syringe using the same, and more particularly, to a needle for injecting a medicinal liquid in the lateral directions of the needle itself penetrating a skin layer so that the medicinal liquid is evenly distributed around a tendon membrane, and a syringe using the same.

### BACKGROUND ART

Various treatments and medical procedures are performed by inserting a needle penetrating the skin layer and by injecting a medicinal liquid into the skin tissue through the needle.

Typically, for rapid painkiller application, a medicinal liquid is injected through the skin layer directly to the blood, or for a skin medical treatment, a method is widely utilized, which includes inserting a needle penetrating the skin layer, and withdrawing the needle from the skin layer while adjusting the withdrawal velocity to uniformly supplying the medicinal liquid to the skin layer.

For example, in a representative cosmetic surgery using diluted botulinum to improve skin beauty, botulinum is injected into the surrounding portions of a wrinkled skin layer to remove wrinkles while contracting muscles.

However, despite such use, because the medicinal liquid is supplied only from the tip of the needle, if botulinum is injected without adjusting the needle withdrawal velocity, botulinum or medicinal liquid is excessively supplied to only a portion of the lower portion of the skin layer, which may lead to side effects.

Representatively, in cosmetic surgery using botulinum, excessive supply of botulinum to the lower portion of the skin layer, that is, the portion adjacent to the bone, leads to side effects, which may cause various inconveniences such as severe swelling of only some portion, severe contraction of only some portion, or the like.

Moreover, in the case of so-called botox injection, in which botulinum is injected, the medicinal liquid is injected through the skin layer into each layer of the skin in various ways depending on the purpose of the medical procedure, and in practice, since the most stable medical procedure is possible only when the medicinal liquid is evenly supplied to the surrounding portions of the tendon membrane, there is a difficulty in that an operator needs to meticulously adjust this.

In order to overcome such drawbacks, a means for injecting a medicinal liquid capable of evenly supplying the medicinal liquid to each layer of the skin has been actively devised, and a method capable of solving these problems is required.

### SUMMARY OF INVENTION

### Technical Problem

The present disclosure has been conceived to address the above-described drawbacks of the related art, and an object of the present disclosure is to inject a medicinal liquid toward the lateral directions of a needle penetrating the skin layer, so that the medicinal liquid is evenly distributed around the tendon membrane.

The objects of the present disclosure are not limited to the aforementioned ones, but other unmentioned objects will be clearly understood from the description below by those skilled in the art.

### Solution to Problem

A needle and a syringe using the same of the present disclosure for achieving the above-described objects includes a needle provided in a device for injecting a medicinal liquid through a skin layer, and including outlets formed along the longitudinal direction of the needle between one end of the needle connected to an accommodating part of the device containing the medicinal liquid and the other end of the needle for penetrating the skin layer, so that the medicinal liquid is evenly injected into a skin tissue in the lower portion of the skin layer, wherein the outlets are formed in a first section between the other end and a location corresponding to a tendon membrane located inside the skin tissue in a state of penetrating the skin layer, and a second section between the location corresponding to the tendon membrane and the one end, respectively, so that the medicinal liquid is evenly injected around the tendon membrane.

In this regard, the outlets are provided with a first hole formed in the first section of between 1.9% and 18.5% from the other end toward the one end.

In this case, the first hole is formed at a location between 0.5 mm and 3.5 mm from the other end.

Meanwhile, there are a plurality of first holes formed at locations spaced apart by the same length with respect to the other end.

Additionally, the first holes form the same angle with each other at the locations spaced apart by the same length with respect to the other end.

Meanwhile, the outlets are provided with a second hole and a third hole formed in the second section of between 25.6% and 86.8% from the other end toward the one end.

In this connection, when the needle used has a length of 19 mm, the second section is between 6.5 mm and 16.5 mm.

And the second hole is formed at a location between 6.5 mm and 11 mm from the other end.

In this case, the third hole is formed at a location between 12 mm and 16.5 mm from the other end.

Meanwhile, when the needle used has a length of 25.4 mm, the second section is between 6.5 mm and 21.5 mm.

Also, the second hole is formed at a location between 6.5 mm and 13.5 mm from the other end.

In this case, the third hole is formed at a location between 14.5 mm and 21.5 mm from the other end.

Meanwhile, when an axis through which the other end is inserted penetrating the skin layer is defined as a first axis, and a central axis of the outlet through which the medicinal liquid moves is defined as a second axis, the outlet is formed such that the first direction and the second direction form a predetermined angle.

Additionally, the outlets are inclined at different angles.

Meanwhile, a needle and a syringe using the same according to an embodiment of the present disclosure include a needle and a cylinder in which a medicinal liquid to be moved through the needle is accommodated.

In this regard, the other end of the needle is closed, so that the medicinal liquid is evenly injected into the skin layer through the outlet of the needle.

Additionally, the outlets are provided with a first hole formed in the first section of between 1.9% and 18.5% from the other end toward the one end.

In this case, the first hole is formed at a location between 0.5 mm and 3.5 mm from the other end.

Additionally, the outlets are provided with a second hole and a third hole formed in the second section of between 25.6% and 86.8% from the other end toward the one end.

In this connection, when the needle used has a length of 19 mm, the second section is between 6.5 mm and 16.5 mm.

And the second hole is formed at a location between 6.5 mm and 11 mm from the other end.

Additionally, the third hole is formed at a location between 12 mm and 16.5 mm from the other end.

Meanwhile, when the needle used has a length of 25.4 mm, the second section is between 6.5 mm and 21.5 mm.

In this regard, the second hole is formed at a location between 6.5 mm and 13.5 mm from the other end.

Additionally, the third hole is formed at a location between 14.5 mm and 21.5 mm from the other end.

### Advantageous Effects

The needle and the syringe using the same of the present disclosure for achieving the above-described objects have the effect of distributing the medicinal liquid evenly to the surrounding portions of the tendon membrane by injecting the medicinal liquid toward the lateral directions of the needle penetrating the skin layer.

Effects of this disclosure are not limited to the effects mentioned above, and other unmentioned effects will be clearly understood from the appended claims by those skilled in the art.

### BRIEF DESCRIPTION OF DRAWINGS

The above-described summary, as well as the detailed description of the preferred embodiments of the present application set forth below, will be better understood when read in conjunction with the accompanying drawings. Preferred embodiments are shown in the drawings for the purpose of illustrating the present disclosure. However, it should be understood that this application is not limited to the precise arrangements and instrumentalities shown.

FIG. 1 is a view for explaining the overall configuration of a needle and a syringe using the same according to an embodiment of the present disclosure;

FIG. 2 is a view for explaining the cross-sectional shape of a needle or a needle of a syringe using the same according to an embodiment of the present disclosure;

FIG. 3 is a view for explaining the locations of the outlets of a needle or the outlets of a needle of a syringe using the same according to an embodiment of the present disclosure;

FIG. 4 is a view for explaining a situation in which a needle or a syringe using the same according to an embodiment of the present disclosure is used;

FIG. 5 is a view for explaining the transverse cross-sectional shape of a needle or a needle of a syringe using the same according to an embodiment of the present disclosure;

FIG. 6 is a view for explaining the overall configuration of a needle having a different length and a syringe using the same according to an embodiment of the present disclosure;

FIG. 7 is a view for explaining the cross-sectional shape of a needle having a different length or a needle of a syringe using the same according to an embodiment of the present disclosure;

FIG. 8 is a view for explaining the locations of the outlets of a needle having a different length or the outlets of a needle of a syringe using the same according to an embodiment of the present disclosure;

FIG. 9 is a view for explaining a situation in which a needle having a different length or a syringe using the same according to an embodiment of the present disclosure is used;

FIG. 10 is a view for explaining the experimental results of the cases where the outlets of a needle and the outlets of a needle of the syringe using the same according to an embodiment of the present disclosure are formed in a parallel state and a perpendicular state;

FIG. 11 is a view for comparing the parallel state and perpendicular state of a needle and a syringe using the same according to an embodiment of the present disclosure;

FIG. 12 is a view for explaining experimental results for confirming the injection velocity of a medicinal liquid at each distance spaced apart from the other end in a parallel state of a needle and a syringe using the same according to an embodiment of the present disclosure;

FIG. 13 is a view for explaining experimental results for confirming the injection velocity of a medicinal liquid at each distance spaced apart from the other end in a perpendicular state of a needle and a syringe using the same according to an embodiment of the present disclosure;

FIG. 14 is a view showing experimental results for comparing medicinal liquid injection velocities when being compared in terms of a hole and an angle of a needle and a syringe using the same according to an embodiment of the present disclosure;

FIG. 15 is a view showing experimental results of kinetic energy according to a injection velocity of a medicinal liquid depending on the hole and the angle of a needle and a syringe using the same according to an embodiment of the present disclosure;

FIG. 16 is a view for explaining quantitative comparison of experimental results according to the pressure of a needle and a syringe using the same according to an embodiment of the present disclosure;

FIG. 17 is a view for explaining pressure simulation results in a parallel state of a needle and a syringe using the same according to an embodiment of the present disclosure;

FIG. 18 is a view for explaining pressure simulation results in a perpendicular state of a needle and a syringe using the same according to an embodiment of the present disclosure; and

FIG. 19 is a view for explaining the base data to be actually applied to a needle and a syringe using the same according to an embodiment of the present disclosure.

### LIST OF REFERENCE SIGNS

A: Predetermined angle
H1: First hole
H2: Second hole
H3: Third hole
S1: First section
S2: Second section
T: Tendon membrane
100: First needle
120: First closed end
200: Cylinder
300: Second needle
320: Second closed end

### BEST MODE

Hereinafter, a preferred embodiment of the present disclosure in which the object of the present disclosure can be realized specifically will be described with reference to the accompanying drawings. In describing the present embodiment, like name and like reference numeral are used to refer to like component, and additional description thereof will be omitted.

The configuration and shape of a needle and a syringe using the same according to an embodiment of the present disclosure will be described first.

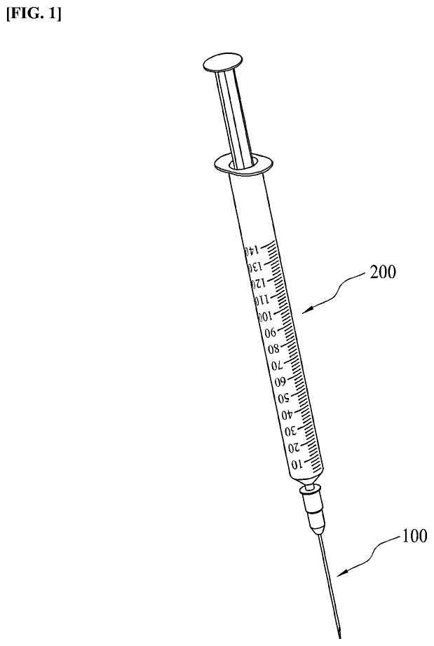

These may be described referring to FIGS. 1 to 9.

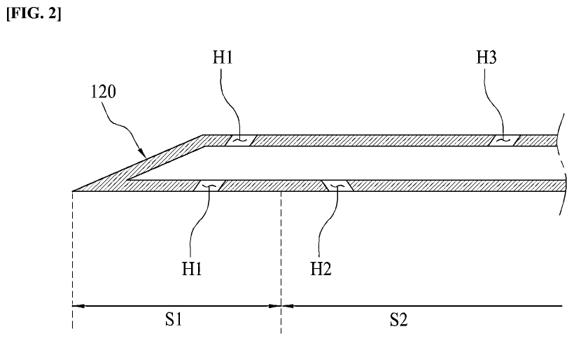

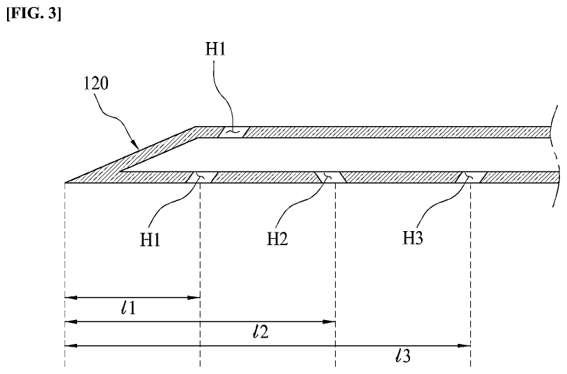

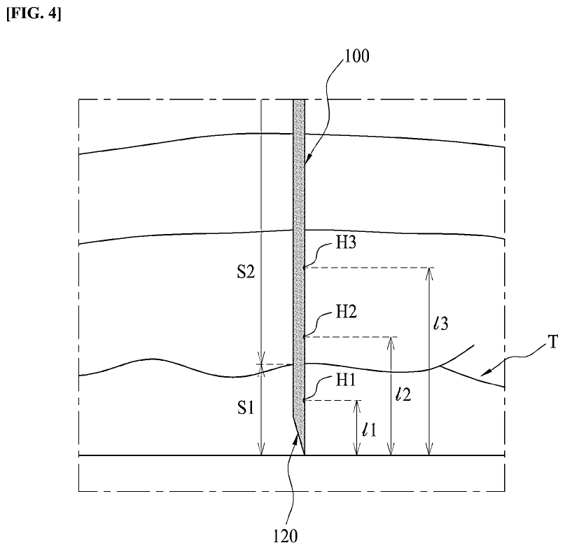

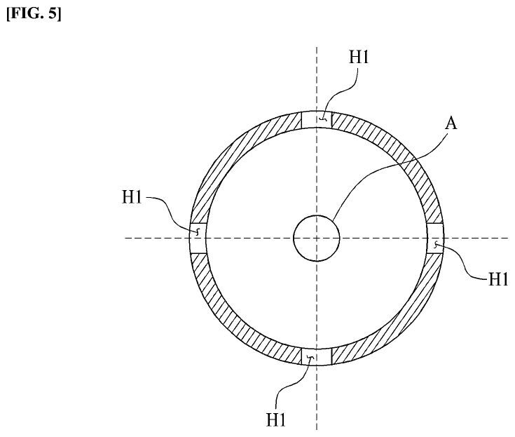

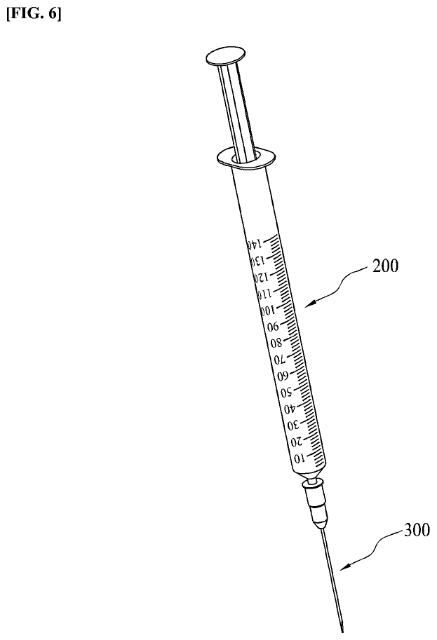

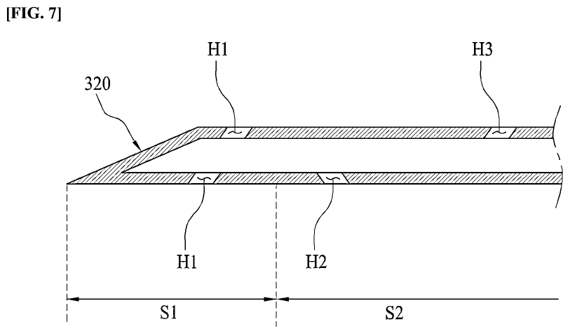

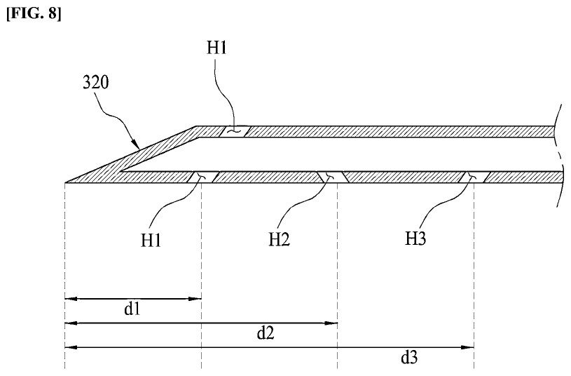

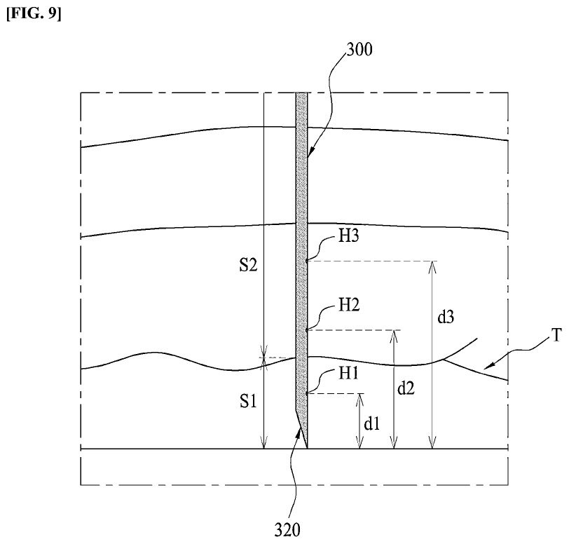

Specifically, FIG. 1 is a view for explaining the overall configuration of a needle and a syringe using the same according to an embodiment of the present disclosure, FIG. 2 is a view for explaining the cross-sectional shape of a needle or a needle of a syringe using the same according to an embodiment of the present disclosure, FIG. 3 is a view for explaining the locations of the outlets of a needle or the outlets of a needle of a syringe using the same according to an embodiment of the present disclosure, FIG. 4 is a view for explaining a situation in which a needle or a syringe using the same according to an embodiment of the present disclosure is used, FIG. 5 is a view for explaining the transverse cross-sectional shape of a needle or a needle of a syringe using the same according to an embodiment of the present disclosure, FIG. 6 is a view for explaining the overall configuration of a needle having a different length and a syringe using the same according to an embodiment of the present disclosure, FIG. 7 is a view for explaining the cross-sectional shape of a needle having a different length or a needle of a syringe using the same according to an embodiment of the present disclosure, FIG. 8 is a view for explaining the locations of the outlets of a needle having a different length or the outlets of a needle of a syringe using the same according to an embodiment of the present disclosure, and FIG. 9 is a view for explaining a situation in which a needle having a different length or a syringe using the same according to an embodiment of the present disclosure is used.

First, as shown in FIG. 1, a syringe using a needle according to an embodiment of the present disclosure may include a needle 100 and a cylinder 200.

In this connection, the needle 100 may be formed thin and long, so that it can be inserted penetrating the skin layer and supply a medicinal liquid toward the skin tissues inside the skin layer.

In this case, the needle 100 may have a length of 19 mm, one end of the needle 100 may communicate with the cylinder 200, and the other end thereof may be inserted to penetrate the skin layer.

On the other hand, the cylinder 200 may be configured such that an internal space can be formed for accommodating the medicinal liquid to be delivered to the needle 100, and the medicinal liquid can be delivered to the needle 100 as the internal pressure increases.

Additionally, the cylinder 200 may be in communication with the one end of the needle 100.

Further, referring to FIGS. 2 to 4 for more detailed description, a plurality of outlets may be formed in the needle 100 along its longitudinal direction.

With such structure, the needle 100 can penetrate the skin layer and evenly supply the medicinal liquid delivered from the cylinder 200 to the inside of the skin tissues through the outlets.

That is, since the plurality of outlets are formed along the longitudinal direction of the needle 100, the medicinal liquid can be evenly supplied to the respective skin layer.

In this connection, as shown in FIG. 2, the needle 100 may be divided into a first section S1 which is from the other end to a location on the needle 100 corresponding to the tendon membrane in a state of penetrating the skin layer, and a second section S2 which is from the first section S1 to the one end of the needle 100.

In this case, each of the first section S1 and the second section S2 may have an outlet formed therein, and a first closed end 120 may be formed at the other end of the needle 100, in order to prevent side effects caused by excessive supply of the medicinal liquid to a location corresponding to the other end of the needle 100 below the skin layer and thus a heavy concentration of the medicinal liquid in a part of the skin layer.

That is, the other end of the needle 100 may be sealed by the first closed end to restrict the medicinal liquid from flowing out of the other end of the needle 100.

Accordingly, the medicinal liquid is supplied to the skin layer only through the outlets so that the medicinal liquid can be evenly supplied along the height of the skin layer.

Meanwhile, at least one first hole H1 may be formed in the first section S1, and a second hole H2 and a third hole H3 may be formed in the second section S2.

That is, when the outer direction of the skin layer is defined as the upper part and the direction toward the location of the bone is defined as the lower part, the first hole H1 may be located in the lower part of the tendon membrane, which is the reference, and the second hole H2 and the third hole H3 may be located in the upper part of the tendon membrane.

In addition, when the length of the needle 100 is 19 mm, the first section S1 may refer to a section between 1.9% and 18.5% from the other end toward the one end, which can be determined based on the average location of the tendon membrane T.

That is, since at least one first hole H1 is formed in the first section S1, the first hole H1 can be formed between 1.9% and 18.5% of 19 mm based on the other end, and it can be formed at a location between 0.5 mm and 3.5 mm spaced from the other end.

Accordingly, the medicinal liquid may be supplied to the inside of the skin layer through the first hole H1 located in the lower part of the tendon membrane T based on the tendon membrane T.

Meanwhile, as shown in FIG. 2, a plurality of first holes H1 may be formed at locations spaced apart by the same length with respect to the other end.

This refers to that a plurality of first holes may be formed in order to inject a medicinal liquid evenly to the surrounding portions of the tendon membrane T.

Meanwhile, the second hole H2 and the third hole H3 may be formed in the second section S2, and the second section S2 may be formed between 25.6% and 86.8% spaced from the other end when the needle 100 is 19 mm in length.

That is, the second section S2 may mean to be between 6.5 mm and 16.5 mm based on the other end.

Additionally, since the second hole H2 and the third hole H3 are formed in the second section S2 as shown in FIG. 2, the second hole H2 and the third hole H3 may be formed to be spaced apart from each other by a predetermined distance in order to supply the medicinal liquid evenly along the height of the skin layer.

That is, the second hole may be formed at a half of the second section S2, i.e., a location between 25.6% and 56.2% spaced from the other end of the needle 100, more specifically, a location between 6.5 mm and 11 mm spaced from the other end of the needle 100.

Additionally, the third hole H3 may be formed in the second section S2 at a location between 56.2% and 86.8% spaced from the other end of the needle 100, more specifically, a location between 12 mm and 16.5 mm spaced from the other end of the needle 100.

Further, although the drawings show that there are formed just one second hole H2 and just one third hole H3, it is obvious that a plurality of second or third holes may be formed according to the manufacturer's intention, and there may be no restriction to the number of the second holes H2 or the third holes H3.

More specifically, as shown in FIG. 3, the first hole H1 may be formed at a location spaced apart from the other end of the needle 100 by l1, and the second hole H2 may be formed at a location spaced apart from the other end of the needle 100 by l2, and the third hole H3 may be formed at a location spaced apart from the other end of the needle 100 by l3.

In this case, l1, l2, and l3 may be included within the previously presented ranges, and in other words, when the length of the needle 100 is 19 mm, l1 may be a value between 0.5 mm and 3.5 mm, l2 may be a value between 6.5 mm and 11 mm, and l3 may be a value between 12 mm and 16.5 mm.

Meanwhile, the first hole H1 to the third hole H3 may be formed offset from each other as shown in FIG. 2, or may be formed on the same axis as shown in FIG. 3.

Moreover, when the longitudinal direction of the needle 100 is designated as a first axis and the direction in which the outlet H1, H2, or H3 is formed is designated as a second axis, that is, the direction in which the medicinal liquid moves through the outlet is designated as a second axis, the first hole H1 to the third hole H3 may be formed such that the first axis and the second axis are inclined to each other in order to adjust the injection velocity and pressure of the medicinal liquid.

Additionally, the plurality of first holes H1 may have the same second axis, and the plurality of first holes H1 to the third hole H3 may have different second axes.

In other words, the first axis and the second axis may be in a non-perpendicular state to each other and in a tilted state to each other.

More specifically, as shown in FIG. 4, in a state in which the needle 100 has penetrated the skin layer, the first section S1 in which the first hole is formed and which is from the other end of the needle 100 to the location of the needle 100 corresponding to the tendon membrane T, and the second section S2 from the location of the needle 100 corresponding to the tendon membrane T to the one end of the needle 100 may be formed such that the first hole H1 and the second hole H2 can be located at the surrounding portions of the tendon membrane T based on the other end of the needle 100.

Further, the first hole H1 may be formed within the first section S1, and the length l1 between the first hole H1 and the other end of the needle 100 may be between 0.5 mm and 3.5 mm, as previously suggested; the second hole H2 may be formed in the second section S2, and the length l2 between the second hole and the other end of the needle 100 may be between 6.5 mm and 11 mm; and the third hole H3 may be formed in the second section S2, and the length l3 between the third hole and the other end of the needle 100 may be between 12 mm and 16.5 mm.

Furthermore, as shown in FIG. 5, a plurality of the first holes H1 may be formed at locations equally spaced apart from the other end to form a predetermined angle A with each other.

In regard to the predetermined angle A, the first holes H1 may be spaced around the center of the needle 100 to form the same angle with each other, or may be spaced to form different angles with each other.

As shown in FIG. 6, a syringe using a needle according to an embodiment of the present disclosure may include a needle 300 and the cylinder 200, and the needle 300 may actually have a similar structure to the needle 100 except for a difference in length.

In this regard, the length of the needle 100 has been described assuming that it is 19 mm, but the needle 300 may be manufactured to have a length of 25.4 mm.

In this case, the needle 300 may have a second closed end 320 formed at the other end of the needle 300 to restrict the movement of the medicinal liquid, similarly to the first closed end 120 described above, as shown in FIG. 7, and with this structure, the medicinal liquid can be evenly supplied to the skin layer from the outlets of the needle 300.

Meanwhile, the needle 300 may have the outlets formed in a different ratio from those of the needle 100.

That is, in the same way as shown in FIG. 7, the first section S1 in which the first hole H1 is located may be formed between 1.9% and 18.5% spaced from the other end of the needle 300, and this may be between 0.5 mm and 3.5 mm in the same way as the needle 100 above.

On the other hand, unlike in the needle 100, the second section S2 may be between 25.6% and 86.8% spaced apart from the other end of the needle 300, which is a section of between 6.5 mm and 21.5 mm.

Accordingly, the second hole H2 may be formed at a location between 6.5 mm and 13.5 mm spaced from the other end of the needle 300, and the third hole H3 may be formed at a location between 14.5 mm and 21.5 mm spaced from the other end of the needle 300, respectively.

That is, as shown in FIG. 8, the distance d1 between the other end of the needle 300 and the first hole H1, the distance d2 between the other end of the needle 300 and the second hole H2, and the distance d3 between the other end of the needle 300 and the third hole H3 may be a range of 0.5 mm to 3.5 mm, a range of 6.5 mm to 13.5 mm, and a range of 14.5 mm to 21.5 mm, respectively, as suggested above.

In addition, as shown in FIG. 9, similarly to the needle 100, in a state in which the needle 300 has penetrated the skin layer, the first hole H1 may be located in the lower part with respect to the tendon membrane T, and the second hole H2 and the third hole H3 are located in the upper part, so that the medicinal liquid can be supplied evenly to the surrounding portions of the tendon membrane T.

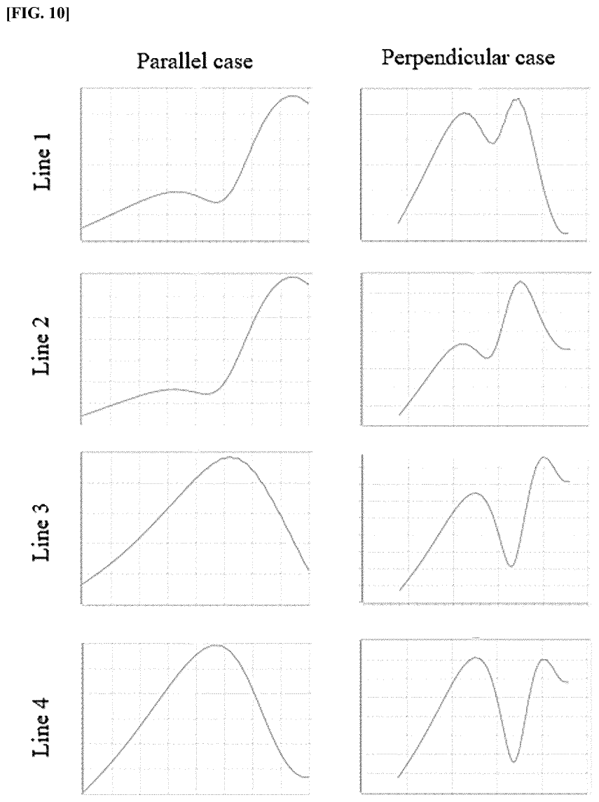

The locations of the outlets formed in the needle and the syringe using the needle according to an embodiment of the present disclosure described above and the experimental results using the needle will be described with reference to FIGS. 10 to 19.

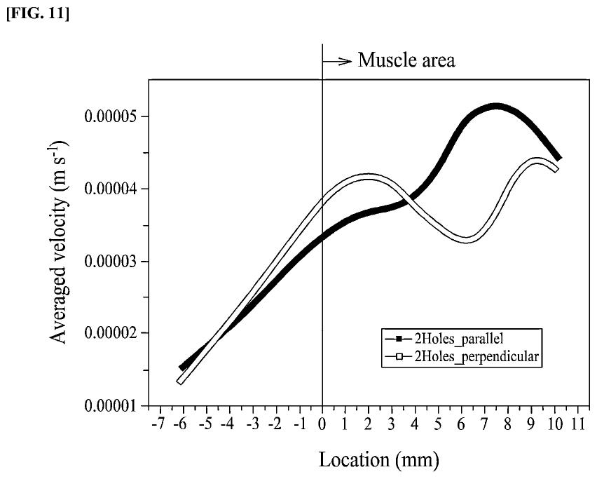

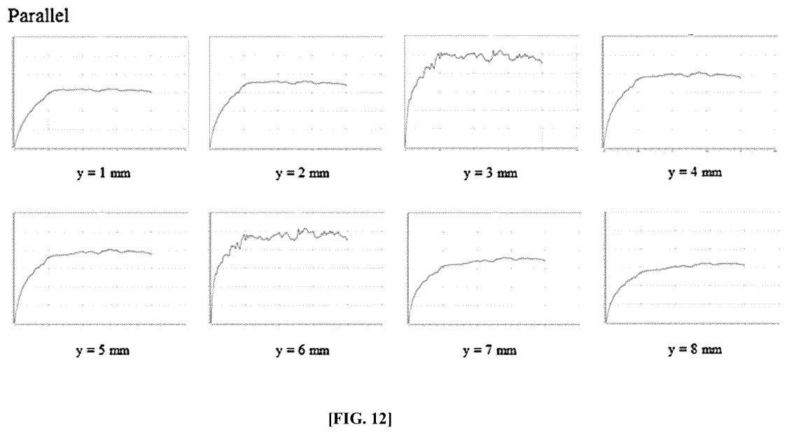

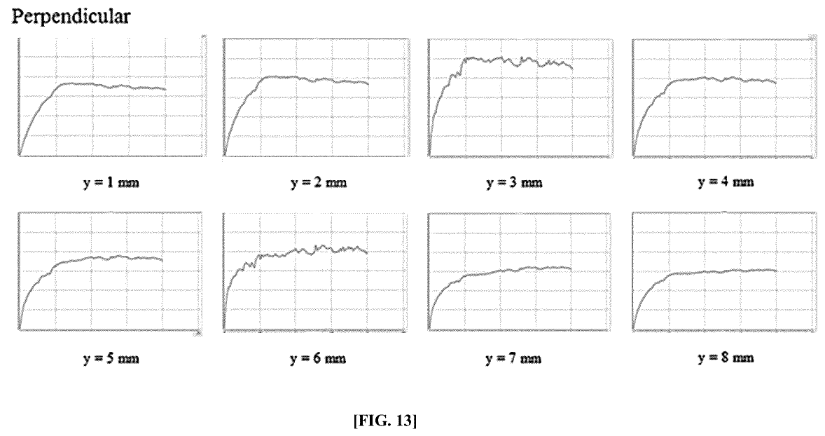

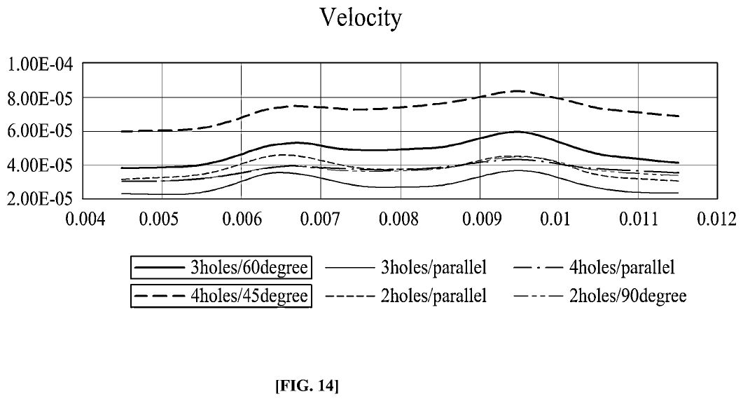

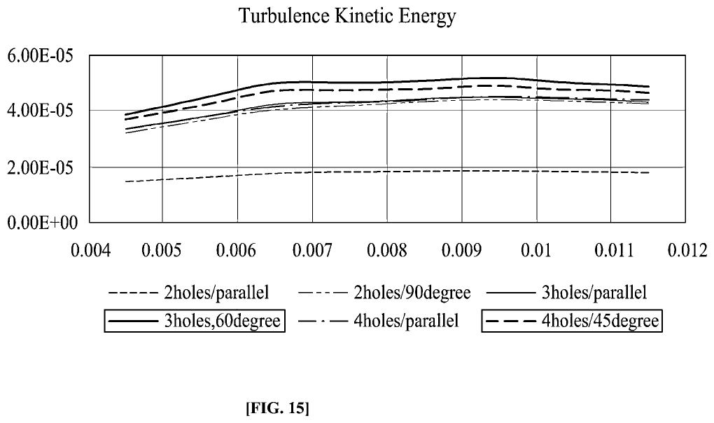

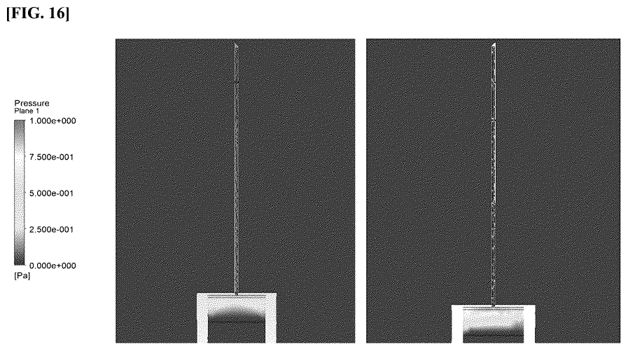

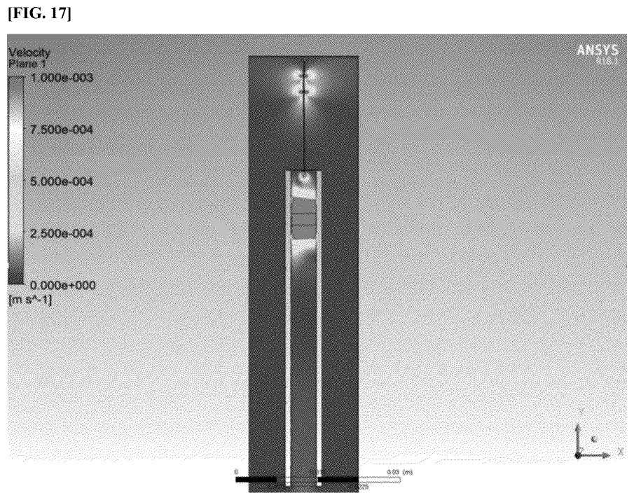

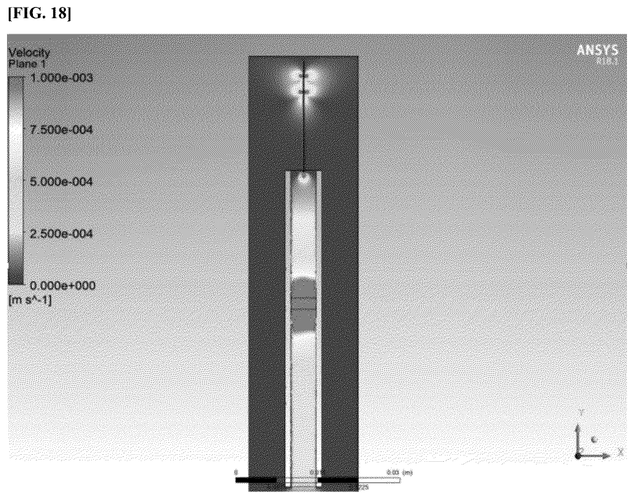

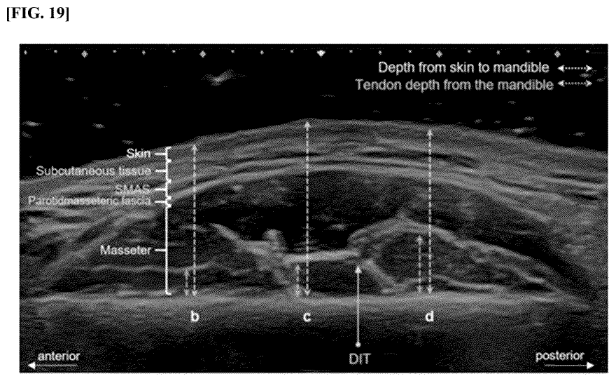

Specifically, FIG. 10 is a view for explaining the experimental results of the cases where the outlets of a needle and the outlets of a needle of the syringe using the same according to an embodiment of the present disclosure are formed in a parallel state and a perpendicular state, FIG. 11 is a view for comparing the parallel state and perpendicular state of a needle and a syringe using the same according to an embodiment of the present disclosure, FIG. 12 is a view for explaining experimental results for confirming the injection velocity of a medicinal liquid at each distance spaced apart from the other end in a parallel state of a needle and a syringe using the same according to an embodiment of the present disclosure, FIG. 13 is a view for explaining experimental results for confirming the injection velocity of a medicinal liquid at each distance spaced apart from the other end in a perpendicular state of a needle and a syringe using the same according to an embodiment of the present disclosure, FIG. 14 is a view showing experimental results for comparing medicinal liquid injection velocities when being compared in terms of a hole and an angle of a needle and a syringe using the same according to an embodiment of the present disclosure, FIG. 15 is a view showing experimental results of kinetic energy according to a injection velocity of a medicinal liquid depending on the hole and the angle of a needle and a syringe using the same according to an embodiment of the present disclosure, FIG. 16 is a view for explaining quantitative comparison of experimental results according to the pressure of a needle and a syringe using the same according to an embodiment of the present disclosure, FIG. 17 is a view for explaining pressure simulation results in a parallel state of a needle and a syringe using the same according to an embodiment of the present disclosure, FIG. 18 is a view for explaining pressure simulation results in a perpendicular state of a needle and a syringe using the same according to an embodiment of the present disclosure, and FIG. 19 is a view for explaining the base data to be actually applied to a needle and a syringe using the same according to an embodiment of the present disclosure.

First, as shown in FIG. 10, the medicinal liquid may be injected at different velocities in a perpendicular state and a parallel state.

Here, the perpendicular state may be an angle formed by the first hole H1 and the second hole H2, by the second hole H2 and the third hole H3, or by the first hole H1 and the third hole H3.

That is, when assuming that the angle is one formed by the first hole H1 and the second hole H2, this may refer to an angle formed between the location of the first hole H1 on a cross section perpendicular to the longitudinal direction of the needle 100 and the location of the second hole H2 with the central axis of the needle 100 as the reference.

More specifically, when the first hole H1 and the second hole H2 are in the parallel state, this may mean that they are located to face each other to form 180 degrees, and when the first hole H1 and the second hole H2 are in the perpendicular state, this may mean that the angle formed by the first hole H1 and the second hole H2 is in a perpendicular state of 90 degrees.

In this regard, as shown in FIG. 10, it can be confirmed that the injection velocities of the medicinal liquid in the parallel state and the perpendicular state are different from each other, and it can be confirmed that the injection velocity of the medicinal liquid varies for each section in the case of the perpendicular state compared to the case of the parallel state.

Additionally, it was confirmed that the injection velocity of the medicinal liquid is different between the case where the outlets are formed in only one line in the longitudinal direction and the case where the outlets are formed in two, three, or four lines.

Meanwhile, referring to FIG. 11 to have a glance at this result, the case where the two holes are in the perpendicular state and the case where they are in the parallel state are compared as shown in FIG. 11, through which it can be confirmed that the injection velocity of the medicinal liquid varies as it gets closer to the bone than to the muscle region, that is, the muscle layer inside the skin layer, and it can be confirmed that as it goes closer to the epidermis of the muscle layer, the injection velocity of the medicinal liquid in the perpendicular state increases, while as it goes farther away from the epidermis of the muscle layer, the injection velocity of the medicinal liquid in the parallel state increases.

Meanwhile, from the result of checking the injection velocity of the medicinal liquid over time with respect to each distance from the other end of the needle 100, it could be confirmed that as shown in FIG. 12, the injection velocity was high in the case of 3 mm and 6 mm in the parallel state, and accordingly, it could be confirmed that the formation of the outlets at the 3 mm and 6 mm positions was appropriate to some extent.

In addition, it could be confirmed that the injection velocity of the medicinal liquid in the perpendicular state was relatively high at 3 mm, but relatively low at 6 mm, compared to that of the parallel state.

That is, based on this, it could be confirmed that the injection velocity of the medicinal liquid in the 3 mm section and the injection velocity of the medicinal liquid in the 6 mm section have relatively high values compared to those in the other sections.

Meanwhile, when compared based on the angle formed by each of the outlets and the number of the outlets, it could be confirmed that the case where three outlets are formed and each of them forms 60 degrees and the case where four outlets are formed and each of them forms 45 degrees have higher injection velocities.

Accordingly, as shown in FIG. 15, it can be confirmed that it is obvious that the case where three outlets are formed and each of them forms 60 degrees and the case where four outlets are formed and each of them forms 45 degrees have higher kinetic energies, as well as the higher injection velocities.

In addition, referring to FIG. 16 to compare the pressure, the figure on the left side of FIG. 16 represents the case where four outlets are formed and each of them forms 45 degrees, and the figure on the right side thereof represents the case where two outlets are formed and each of them forms 180 degrees.

When comparing these cases to each other, it could be confirmed that the case where two outlets are formed and each of them forms 180 degrees has a higher pressure than the case where four outlets are formed and each of them forms 45 degrees.

Further, as shown in FIGS. 17 and 18 for direct comparison, it could be confirmed that the pressure distribution in the perpendicular state of the perpendicular state and the parallel state is formed relatively higher, and it could be confirmed that when the outlets are formed in different directions, the injection velocity of the medicinal liquid and the velocity at which the medicinal liquid is distributed evenly are increased.

Meanwhile, as shown in FIG. 19, it can be confirmed that the first hole H1, the second hole H2, and the third hole H3 have different locations as b, c, and d of the tendon membrane T, respectively, and therefore, the section between 18.5% and 25.6% spaced apart from the other end of the needle 100, which is excluded from the first section S1 and the second section S2, may be a location corresponding to the tendon membrane T based on the predetermined distances in the range of the location of the tendon membrane T.

Accordingly, the first section S1 and the second section S2 may have an effect of injecting the medicinal liquid into the surrounding portions of the tendon membrane T, and an effect of preventing the injection of the medicinal liquid into the tendon membrane T which might cause facial paralysis.

As described above, the preferred embodiments according to the present disclosure have been discussed, and it is obvious to those skilled in the art that the present disclosure may be embodied in other specific forms than the above-described embodiments without departing from its technical idea or scope. Therefore, it should be noted that the embodiments described above are to be regarded as illustrative rather than restrictive, and thus the present disclosure is not limited to the above description, but its modifications and variation may fall within the scope of the following claims and their equivalents.

1-25. (canceled)

26. A needle provided in a device for injecting a medicinal liquid through a skin layer, the needle comprising:
outlets formed along the longitudinal direction of the needle between a first end of the needle connected to an accommodating part of the device containing the medicinal liquid and a second end of the needle configured to penetrate the skin layer, so that the medicinal liquid can be evenly injected into a skin tissue in the lower portion of the skin layer,
wherein the outlets are formed in a first section between the second end and a location corresponding to a tendon membrane located inside the skin tissue when the needle is in a state of penetrating the skin layer, and in a second section between the location corresponding to the tendon membrane and the first end, respectively, so that the medicinal liquid is evenly injected around the tendon membrane.

27. The needle of claim 26, wherein the outlets are provided with a first hole formed in the first section of between 1.9% and 18.5% from the second end toward the first end.

28. The needle of claim 27, wherein the first hole is formed at a location between 0.5 mm and 3.5 mm from the second end.

29. The needle of claim 27, wherein there are a plurality of first holes formed at locations spaced apart by the same length with respect to the second end.

30. The needle of claim 29, wherein the first holes form the same angle with each other at the locations spaced apart by the same length with respect to the second end.

31. The needle of claim 26, wherein the outlets are provided with a second hole and a third hole formed in the second section of between 25.6% and 86.8% from the second end toward the first end.

32. The needle of claim 31, wherein when the needle used has a length of 19 mm, the second section is between 6.5 mm and 16.5 mm.

33. The needle of claim 32, wherein the second hole is formed at a location between 6.5 mm and 11 mm from the second end.

34. The needle of claim 32, wherein the third hole is formed at a location between 12 mm and 16.5 mm from the second end.

35. The needle of claim 31, wherein when the needle used has a length of 25.4 mm, the second section is between 6.5 mm and 21.5 mm.

36. The needle of claim 35, wherein the second hole is formed at a location between 6.5 mm and 13.5 mm from the second end.

37. The needle of claim 35, wherein the third hole is formed at a location between 14.5 mm and 21.5 mm from the second end.

38. The needle of claim 26, wherein when an axis through which the second end is configured to be inserted to penetrate the skin layer is defined as a first axis, and a central axis of the outlet through which the medicinal liquid is configured to move is defined as a second axis, the outlet is formed such that the first direction and the second direction form a predetermined angle.

39. The needle of claim 38, wherein the outlets are inclined at different angles.

40. A syringe comprising:
a needle according to claim 26; and
a cylinder in which a medicinal liquid to be moved through the needle can be accommodated.

41. The syringe of claim 40, wherein the second end of the needle is closed, so that the medicinal liquid can be evenly injected into the skin layer through the outlet of the needle.

42. The syringe of claim 40, wherein the outlets are provided with a first hole formed in the first section of between 1.9% and 18.5% from the second end toward the first end.

43. The syringe of claim 42, wherein the first hole is formed at a location between 0.5 mm and 3.5 mm from the second end.

44. The syringe of claim 40, wherein the outlets are provided with a second hole and a third hole formed in the second section of between 25.6% and 86.8% from the second end toward the first end.

45. The syringe of claim 44, wherein when the needle used has a length of 19 mm, the second section is between 6.5 mm and 16.5 mm.

46. The syringe of claim 45, wherein the second hole is formed at a location between 6.5 mm and 11 mm from the second end.

47. The syringe of claim 45, wherein the third hole is formed at a location between 12 mm and 16.5 mm from the second end.

48. The syringe of claim 44, wherein when the needle used has a length of 25.4 mm, the second section is between 6.5 mm and 21.5 mm.

49. The syringe of claim 48, wherein the second hole is formed at a location between 6.5 mm and 13.5 mm from the second end.

50. The syringe of claim 48, wherein the third hole is formed at a location between 14.5 mm and 21.5 mm from the second end.
